(19) United States
(12) Patent Application Publication
(10) Pub. No.: US 2008/0305132 A1
(43) Pub. Date: Dec. 11, 2008

(54) Cosmetic Patch for Skin Treatment

(76) Inventor: Mihal Jozef Karol, Halifax (GB)

Correspondence Address: TREGO, HINES & LADENHEIM, PLLC, 9300 HARRIS CORNERS PARKWAY, SUITE 210, CHARLOTTE, NC, 28269-3797, US

(21) Appl. No.: 11/720,412

(22) PCT Filed: Nov. 29, 2005
(86) PCT No.: PCT/GB05/04550

§ 371 Date: Jan. 14, 2008

(30) Foreign Application Priority Data
Nov. 30, 2004 (GB) 0426255.6

(51) Int. Cl. A61K 8/89; A61K 8/02; A61K 9/70; A61P 17/02; A61K 31/765; A61Q 19/08

(52) U.S. Cl. 424/401; 424/78.02; 424/78.06; 424/443

(57) ABSTRACT

There is provided a treatment product (1) comprising a patch (3), said patch (3) comprising a polysiloxane as a treatment agent, and wherein the patch (3) is arranged such that it can be removeably adhered to a user's skin. Also provided are other treatment products, the use of polysiloxane for manufacturing a medicament and the use of polysiloxane to treat a cutaneous condition.

### FIELD OF THE INVENTION

The present invention relates to products for skin treatment and to the use of such products, particularly in cosmetic treatments.

### BACKGROUND TO THE INVENTION

The use of cosmetic products to improve the appearance of skin is well known and such products include compositions intended to reduce the appearance of fine lines and wrinkles.

EP1136064 for example discloses a topical composition comprising spherical and/or substantially spherical optical diffuser particles and cross linked silicone elastomer in a vehicle. The silicone elastomer evens out lines and wrinkles to allow the spherical particles to reflect light and effectively function as an optical diffuser reducing the appearance of the lines and wrinkles. This composition must though be spread onto the skin by a user and relies on the use of spherical particles.

US20020131948 discloses a composition for reducing or removing wrinkles and/or fine lines comprising at least one grafted silicone polymer which comprises a polysiloxane portion and a portion composed of a non-silicone organic chain. Again, this composition must be spread onto the skin by a user and it is also reliant on the use of a grafted polymer.

WO9926571 relates to transdermal delivery devices containing a pressure-sensitive adhesive incorporating a polydiorganosiloxane polymer to regulate the adhesive properties. These devices can be used to apply drugs and cosmetics but are reliant on the use of an adhesive such as acrylic copolymers in addition to a drug or cosmetic.

There thus remains a need for alternative treatment products. Accordingly, the present invention thus aims to address at least one disadvantage associated with the prior art whether discussed herein or otherwise.

### SUMMARY OF THE INVENTION

According to a first aspect of the present invention there is provided a treatment product comprising a patch, said patch comprising a polysiloxane as a treatment agent, and wherein the patch is arranged such that it can be removeably adhered to a user's skin.

Preferably, the invention provides a treatment product comprising a patch for use in the treatment of a cutaneous condition, said patch comprising a polysiloxane as a treatment agent for the cutaneous condition, and wherein the patch is arranged such that it can be removeably adhered to a user's skin.

The patch may be arranged for use to treat one or more of: wrinkles and/or fine lines; scars, preferably post-operative scars; acne; bites; skin allergies; and sun burn.

Suitably, the patch comprises a polysiloxane as both a treatment agent, preferably a treatment agent for a cutaneous condition, and as an adhesive to adhere the patch to a user's skin.

The patch may have the advantage that it can be applied more quickly and/or cleanly than a liquid composition which must be rubbed onto the skin by a user. The patch may have the advantage that it can be quickly and/or cleanly removed by a user after a desired period of time.

Surprisingly, it has been found that a polysiloxane may itself have sufficient adhesive properties to adhere a patch to skin and that there may thus be no need for additional adhesives. The patch may thus have the advantage that it is simple in construction with the treatment (active) agent also serving as the adhesive. Accordingly, the patch may not require the use of additional adhesives which may have an adverse effect on the skin.

The patch may not employ any treatment agent other than a polysiloxane. For example, the patch may be arranged to treat wrinkles and/or fine lines and may not comprise any treatment agent other than a polysiloxane. Alternatively, the patch may comprise one or more additional treatment agents. The use of additional treatment agent(s) may improve the patch's ability to treat one or more of: wrinkles and/or fine lines; scars, preferably post-operative scars; acne; insect bites; skin allergies; or sunburn.

Suitably, said additional treatment agents comprise extracts and/or oils selected from the group consisting of: Apricot Kernel; Avacado; Calendula; Carrot; Evening Primrose; Grape Seed; Macadamia; Olive; Peach Kernel; Rosehip; Sesame; Sweet Almond; Sunflower; Wheat Germ; Jojoba; Basil; Benzoin; Bergamot; Black Pepper; Cajeput; Cedarwood; Chamomile; ‘Clary’ sage; Eucalyptus; Fennel; Frankincense; Geranium; Ginger; Grapefruit; Jasmine absolute; Juniper Berry; Lavender; Lemon; Lemon Grass; Mandarin; Melissa; Myrrh; Neroli (Orange Blossom); Niaouli; Orange (sweet); Palmarosa; Patchouli; Peppermint; Petitgrain; Rose; Rosemary; Rosewood; Sandalwood; Tea Tree; Cucumber; vitamin C; vitamin E; Thiamine, Zinc, Selenium; Comfrey; Elderflower; Marshmallow; Orange Blossom; Rose; Violet; Borage; Aloe Vera; Honey; Peppermint (oil); Yarrow; Marigold (Calendula varieties); Chamomile; Almond; Argan; Thyme; Vetivert; Witch Hazel and Ylang Ylang.

The patch may for example comprise chamomile and/or witch hazel as an additional treatment agent.

Suitably, the treatment product comprises a cosmetic product.

Suitably, the patch is arranged to treat wrinkles and/or fine lines in the skin. The patch may be arranged to reduce the appearance of wrinkles and/or fine lines. The patch may be arranged to cause the size of wrinkles and/or fine lines to physically reduce. This may be due to a chemical interaction occurring at the skin surface. The patch may thus do more than merely deliver a composition which “fills in” wrinkles to give a smooth surface to the skin.

Suitably, the patch is arranged to be left on the skin for between 10 minutes and 24 hours, for example for between 4 and 10 hours. The patch may thus be left on a user's skin overnight to provide a treating effect. Alternatively, in use, the patch may be left on the skin for around 30 to 45 minutes to provide a treating effect.

Suitably, the patch comprises a polysiloxane sheet. The sheet may have a thickness of between 0.1 and 5 mm, for example of around 1 mm.

Suitably, the polysiloxane comprises a gel which may be a viscous gel. Suitably, the polysiloxane comprises an oil. Preferably, the patch comprises a polysiloxane gel and oil combination.

Suitably, the patch comprises a polydiorganosiloxane. Preferably, the patch comprises polydimethylsiloxane.

Suitably, the treatment product is substantially free of grafted silicone polymer comprising a polysiloxane portion and a portion composed of a non-silicone organic chain.

Suitably, the patch comprises a combination of polysiloxanes. Said polysiloxanes may be cured. Suitably, the patch comprises two or more, preferably three polysiloxanes. Preferably, the patch comprises two polysiloxanes which are cured to form a gel and one further polysiloxane in oil form. Suitably, said oil is distributed homogeneously within the gel.

The patch may comprise one or more of: polysilicone-1; polysilicone-2; and dimethicone as classified by the INCI list. The patch may comprise polysilicone-1, polysilicone-2 and dimethicone. The polysiloxane sheet may consist of these components. Surprisingly, this combination may be particularly effective for treating cutaneous conditions.

Suitably, the patch comprises polysilicone-1 and polysilicone-2 cured to form a gel and dimethicone in the form of an oil.

The patch may comprise silicone oil and/or polydimethylvinylsiloxane and/or polydimethylhydrogensiloxane.

Preferably, the patch comprises silicone oil, polydimethylvinylsiloxane and polydimethylhydrogensiloxane. The polysiloxane sheet may consist of these components.

Suitably, the patch comprises polydimethylsiloxane (dimethicone) and/or polydimethylvinylsiloxane and/or polydimethylhydrogensiloxane.

Preferably, the patch comprises polydimethylsiloxane, polydimethylvinylsiloxane and polydimethylhydrogensiloxane. The polysiloxane sheet may consist of these components. Surprisingly, it has been found that the combination of these three polysiloxanes may be particularly effective in the treatment of cutaneous conditions. The combination may be particularly effective in the treatment of wrinkles and/or fine lines.

Preferably, the polysiloxane of the patch comprises dimethicone (silicone oil) in an amount of between 10% and 50% by weight, preferably between 20% and 40%, more preferably between 25% and 35%, for example around 30% by weight.

Preferably, the polysiloxane of the patch comprises polydimethylvinylsiloxane in an amount of between 15% and 55% by weight, preferably between 25% and 45%, more preferably between 30% and 40%, for example around 35% by weight.

Preferably, the polysiloxane of the patch comprises polydimethylhydrogensiloxane in an amount of between 15% and 55% by weight, preferably between 25% and 45%, more preferably between 30% and 40%, for example around 35% by weight.

The polysiloxane of the patch may comprise dimethicone (silicone oil), polydimethylvinylsiloxane and polydimethylhydrogensiloxane in a combined amount of 100% by weight.

Suitably, the patch comprises a support member carrying the polysiloxane. Suitably, the support member comprises a sheet which is suitably a plastics sheet. The support member may for example comprise a vinyl or polyurethane film.

Suitably, a first side of the polysiloxane sheet is arranged, in use, to be applied to the skin. Suitably, the support member is arranged to lie against an opposed second side of the polysiloxane sheet to substantially cover it.

Suitably, the patch consists of a polysiloxane sheet and a support member.

Suitably, the treatment product comprises a backing member carrying the patch. Suitably, the backing member comprises a sheet which is suitably a plastics sheet.

Suitably, the backing member is arranged to lie against a first side of a polysiloxane sheet and the support member against an opposed second side of the polysiloxane sheet.

The backing member may substantially prevent material adhering to the first side of the polysiloxane sheet and the adhesive nature of the sheet may thus be substantially preserved.

Suitably, the polysiloxane sheet is arranged to adhere more strongly to the support member than to the backing member. The patch may thus be arranged to peel from the backing member such that it can be applied to the skin.

The support member may be arranged to minimise the risk of the polysiloxane sheet tearing or breaking up when the patch is separated from the backing member and/or when subsequently removed from a user's skin.

Suitably, the patch is substantially crescent shaped. The patch may thus be arranged to locate under the eye or mouth of a user. The patch may thus be suitable for treating wrinkles and/or fine lines around a user's eye or mouth.

The treatment product may comprise a plurality of patches. The patches may be substantially identical and may be carried by one backing member. Suitably, the treatment product comprises two patches.

Suitably, the treatment product comprises a container in which the or each patch is enclosed prior to use. The container may comprise a sealed packet, which may be a re-sealable sachet.

According to a second aspect of the present invention there is provided a treatment product comprising polydimethylsiloxane (dimethicone) and/or polysilicone-1 and/or polysilicone-2.

Preferably, according to the invention there is provided a treatment product for use in the treatment of a cutaneous condition, said product comprising polydimethylsiloxane (dimethicone) and/or polysilicone-1 and/or polysilicone-2.

Preferably, according to the invention there is provided a treatment product comprising silicone oil and/or polydimethylvinylsiloxane and/or polydimethylhydrogensiloxane.

Preferably, according to the invention there is provided a treatment product comprising polydimethylsiloxane and/or polydimethylvinylsiloxane and/or polydimethylhydrogensiloxane.

Preferably, according to the invention there is provided a treatment product for use in the treatment of a cutaneous condition, said product comprising polydimethylsiloxane and/or polydimethylvinylsiloxane and/or polydimethylhydrogensiloxane.

Suitably, the treatment product comprises polydimethylsiloxane, polydimethylvinylsiloxane and polydimethylhydrogensiloxane.

The treatment product may comprise a cosmetic product. Suitably, the product is arranged for use in the treatment of wrinkles and/or fine lines.

The treatment product may comprise a patch. Alternatively, the treatment product may comprise a liquid.

The treatment product may comprise any feature as described in relation to the first aspect.

According to a third aspect of the present invention there is provided the use of a polysiloxane composition for the manufacture of a medicament for treating a cutaneous condition.

Preferably, the cutaneous condition comprises wrinkles and/or fine lines. Alternatively, the cutaneous condition may comprise one or more of: scars, preferably post-operative scars; acne; bites; skin allergies; and sun burn.

Suitably, the medicament comprises a treatment product according to the first or second aspect.

Suitably, the medicament comprises a patch comprising the polysiloxane composition.

Suitably, the polysiloxane composition comprises a polydiorganosiloxane. Suitably, the composition comprises polydimethylsiloxane.

Suitably, the medicament is substantially free of grafted silicone polymer comprising a polysiloxane portion and a portion composed of a non-silicone organic chain.

Suitably, the polysiloxane composition comprises polydimethylsiloxane (dimethicone) and/or polysilicone-1 and/or polysilicone-2.

Suitably, the polysiloxane composition comprises silicone oil and/or polydimethylvinylsiloxane and/or polydimethylhydrogensiloxane.

Suitably, the polysiloxane composition comprises polydimethylsiloxane and/or polydimethylvinylsiloxane and/or polydimethylhydrogensiloxane.

Preferably, the polysiloxane composition comprises polydimethylsiloxane and polydimethylvinylsiloxane and polydimethylhydrogensiloxane.

The polysiloxane composition and/or the medicament may comprise any feature as described in relation to the first or second aspect.

According to a fourth aspect of the present invention there is provided the use of a polysiloxane for the treatment of a cutaneous condition.

Preferably, according to the invention there is provided the use of a polysiloxane for the treatment of one or more of: wrinkles and/or fine lines; scars, preferably post-operative scars; acne; bites; skin allergies; and sun burn.

Preferably, the method comprises the use of polysiloxane as a cosmetic product. Suitably, the method comprises applying the polysiloxane to the skin.

Preferably, according to the invention there is provided the use of a polysiloxane as a cosmetic product for the treatment of wrinkles and/or fine lines.

Suitably, the cosmetic product comprises a treatment product according to the first or second aspect.

Suitably, the polysiloxane is employed to treat wrinkles and/or fine lines in the skin. The polysiloxane may be employed to reduce the appearance of wrinkles and/or fine lines. The polysiloxane may be employed to cause the size of wrinkles and/or fine lines to physically reduce.

Suitably, the polysiloxane is employed to adhere the product to a user's skin.

Preferably, the polysiloxane is employed to treat wrinkles and/or fine lines in the skin and to adhere the product to a user's skin.

Suitably, the polysiloxane is used in a patch.

Suitably, the polysiloxane comprises a polydiorganosiloxane. The polysiloxane may comprise polydimethylsiloxane.

Suitably, the polysiloxane is used in a product which is substantially free of grafted silicone polymer comprising a polysiloxane portion and a portion composed of a non-silicone organic chain.

Suitably, the polysiloxane composition comprises polydimethylsiloxane (dimethicone) and/or polysilicone-1 and/or polysilicone-2.

Suitably, the polysiloxane comprises silicone oil and/or polydimethylvinylsiloxane and/or polydimethylhydrogensiloxane. Suitably, the polysiloxane comprises polydimethylsiloxane and/or polydimethylvinylsiloxane and/or polydimethylhydrogensiloxane.

Suitably, the polysiloxane comprises polydimethylsiloxane and/or polydimethylvinylsiloxane and/or polydimethylhydrogensiloxane.

Preferably, the polysiloxane comprises polydimethylsiloxane and polydimethylvinylsiloxane and polydimethylhydrogensiloxane.

The polysiloxane and/or cosmetic product may comprise any feature as described in relation to the first or second aspect.

### BRIEF DESCRIPTION OF THE DRAWINGS

The present invention will now be illustrated by way of example with reference to the accompanying drawings in which:

FIG. 1 is a plan view of a treatment product.

FIG. 2 is a cross section along line A-A of FIG. 1.

### DETAILED DESCRIPTION OF THE PREFERRED EMBODIMENTS

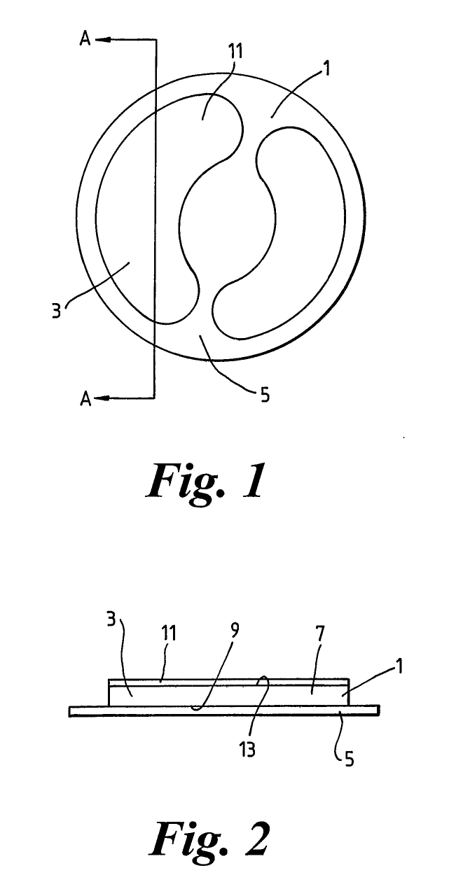

As illustrated by FIG. 1 a treatment product 1 for treating a cutaneous condition, such as wrinkles and/or fine lines in the skin, comprises a patch 3. In the illustrated case the product 1 comprises two patches 3 upon a single backing member 5 which comprises a plastics sheet.

Each patch 3 comprises a polysiloxane sheet 7 comprising polydimethylsiloxane, polydimethylvinylsiloxane and polydimethylhydrogensiloxane.

The polysiloxane used in the polysiloxane sheet is produced by mixing 30 wt % polydimethylsiloxane (silicone oil), 35 wt % polydimethylvinylsiloxane and 35 wt % polydimethylhydrogensiloxane and curing in the presence of a platinum catalyst. This creates a homogious gel (formed from cured polydimethylvinylsiloxane and polydimethylhydrogensiloxane) and oil (polydimethylsiloxane) combination. In an alternative embodiment curing may be achieved using UV radiation or heat without the use of a platinum catalyst.

A first side 9 of the polysiloxane sheet 7 is arranged, in use, to be applied against a user's skin. The polysiloxane sheet 7 is tacky and thus adheres to the user's skin. The polysiloxane sheet 7 also serves as an active treatment agent and can cause a reduction in wrinkles and/or fine lines in the skin.

The backing member 5 covers the first side 9 of the sheet 7 such that it substantially prevents material adhering to the sheet 7 and the sheet 7 thus substantially retains its adhesiveness until a user wishes to use it.

A support member 11 comprising a plastics sheet is mounted to a second side 13 of the polysiloxane sheet. The support member 11 adds structural support to the patch 3 and reduces the risk of the polysiloxane sheet 7 tearing or breaking up in use.

The illustrated patches 3 are intended to be applied around the eyes and are crescent shaped to provide a good fit.

The product further comprises a polypropylene/polyester laminate re-sealable sachet (not illustrated) in which the patches 3 are sealed prior to use.

In use, a user opens the sachet and peels a patch 3 from the backing member 5 and applies it below an eye with the first side 9 of the sheet 7 against the skin. The user then does the same with the other patch 3 for the other eye. The patches 3 are retained on the skin by the tacky nature of the polysiloxane sheet 7. The patches 3 are then left in situ for around 30 minutes to 8 hours and then the user peels them from the skin. Although the patches are tacky enough to adhere to the skin they only weakly adhere and may thus be removed easily without causing the user discomfort.

It has been observed that use of treatment products 1 as described above can give a visible reduction in wrinkles around the eyes.

It will be appreciated that preferred embodiments of the present invention may provide a treatment product which allows for convenient treatment of wrinkles and/or other cutaneous conditions.

Although the description above describes a product for use around a user's eye, embodiments of the present invention can be used whenever needed on a user's skin.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(S). The invention extends to any novel one, or any novel combination, of the features disclosed in this specification (including any accompanying claims, abstract and drawings), or to any novel one, or any novel combination, of the steps of any method or process so disclosed.

1-27. (canceled)

28. A treatment product comprising a patch, said patch comprising a polysiloxane as a treatment agent, and wherein the patch is arranged such that it can be removeably adhered to a user's skin.

29. A treatment product according to claim 28, wherein the patch comprises a polysiloxane gel and oil combination.

30. A treatment product according to claim 28, wherein the patch comprises one or more of: polysilicone-1; polysilicone-2; and dimethicone.

31. A treatment product according to claim 28, wherein the patch comprises polydimethylsiloxane (dimethicone) and/or polydimethylvinylsiloxane and/or polydimethylhydrogensiloxane.

32. A treatment product according to claim 28, wherein the patch comprises polydimethylsiloxane polydimethylvinylsiloxane and polydimethylhydrogensiloxane.

33. A treatment product according to claim 28, wherein the product comprises a patch for use in the treatment of a cutaneous condition, said patch comprising a polysiloxane as a treatment agent for the cutaneous condition.

34. A treatment product according to claim 28, wherein the patch is arranged for use to treat one or more of: wrinkles and/or fine lines; scars; acne; bites; skin allergies; and sun burn.

35. A treatment product according to claim 28, wherein the patch is arranged to treat wrinkles and/or fine lines.

36. A treatment product according to claim 28, wherein the patch comprises a polysiloxane as both a treatment agent and as an adhesive to adhere the patch to a user's skin.

37. A treatment product according to claim 28, wherein the patch does not employ any treatment agent other than a polysiloxane.

38. A treatment product according to claim 28, wherein the patch comprises one or more additional treatment agents comprising extracts and/or oils selected from the group consisting of: Apricot Kernel; Avacado; Calendula; Carrot; Evening Primrose; Grape Seed; Macadamia; Olive; Peach Kernel; Rosehip; Sesame; Sweet Almond; Sunflower; Wheat Germ; Jojoba; Basil; Benzoin; Bergamot; Black Pepper; Cajeput; Cedarwood; Chamomile; ‘Clary’ sage; Eucalyptus; Fennel; Frankincense; Geranium; Ginger; Grapefruit; Jasmine absolute; Juniper Berry; Lavender; Lemon; Lemon Grass; Mandarin; Melissa; Myrrh; Neroli (Orange Blossom); Niaouli; Orange (sweet); Palmarosa; Patchouli; Peppermint; Petitgrain; Rose; Rosemary; Rosewood; Sandalwood; Tea Tree; Cucumber; vitamin C; vitamin E; Thiamine, Zinc, Selenium; Comfrey; Elderflower; Marshmallow; Orange Blossom; Rose; Violet; Borage; Aloe Vera; Honey; Peppermint (oil); Yarrow; Marigold (Calendula varieties); Chamomile; Almond; Argan; Thyme; Vetivert; Witch Hazel and Ylang Ylang.

39. A treatment product according to claim 28, wherein the patch comprises a polysiloxane sheet.

40. A treatment product according to claim 28, wherein the patch comprises a polydiorganosiloxane.

41. A treatment product according to claim 28, wherein the patch comprises a combination of polysiloxanes.

42. A treatment product according to claim 28, wherein the patch comprises a support member carrying the polysiloxane.

43. A treatment product according to claim 28, wherein the treatment product comprises a backing member carrying the patch.

44. A treatment product according to claim 28, wherein the patch is substantially crescent shaped.

45. A treatment product comprising polydimethylsiloxane (dimethicone) and/or polysilicone-1 and/or polysilicone-2.

46. A product according to claim 45, said product being a treatment product for use in the treatment of a cutaneous condition.

47. A treatment product comprising polydimethysiloxane and/or polydimethylvinylsiloxane and/or polydimethylhydrogensiloxane.

48. A product according to claim 47, said product being a treatment product for use in the treatment of a cutaneous condition.

49. A treatment product according to claim 47, wherein the treatment product comprises polydimethylsiloxane, polydimethylvinylsiloxane and polydimethylhydrogensiloxane.

50. The use of a polysiloxane composition for the manufacture of a medicament for treating a cutaneous condition.

51. A method according to claim 50, wherein the medicament comprises a treatment product comprising a patch, said patch comprising a polysiloxane as a treatment agent, and wherein the patch is arranged such that it can be removeably adhered to a user's skin.

52. The use of polysiloxane for the treatment of a cutaneous condition.

53. A method according to claim 52, wherein the method comprises the use of a polysiloxane as a cosmetic product for the treatment of wrinkles and/or fine lines.

54. A method according to claim 52, wherein the method comprises the use of polysiloxane as a cosmetic product and wherein the cosmetic product comprises a treatment product comprising a patch, said patch comprising a polysiloxane as a treatment agent, and wherein the patch is arranged such that it can be removeably adhered to a user's skin.
